SEQUENCE LISTING (6 sequences)

SEQ ID NO: 1 - DNA, 2559 bp, Arabidopsis thaliana
atgggagagaaagcgaaagcaaatgtgttagtcttctcatttccgatacaaggtcacataaaccctctcctccaattctcaaaacgcctactctctaaaaacgtcaacgtcacattcctcaccacttcctccacccacaactccatcctccgccgtgccatcaccggcggagccactgctcttcctctctcttttgtccccattgacgatggattcgaggaagatcacccatctacggacacatctcccgactacttcgcaaagttccaagaaaacgtatctcgaagcctctcagagcttatctcctcgatggacccaaaaccaaacgccgtcgtttacgactcgtgcctgccttatgtcctcgacgtttgccggaaacatcctggcgttgctgcggcgtcgtttttcactcagtcctccaccgtgaacgcgacctatattcatttcttgcgtggagagtttaaggagtttcaaaatgatgtcgttttgcctgcaatgcctccgctgaagggtaatgacttaccggtgtttctgtacgataacaatctctgccggccgttgtttgagctcattagtagccagttcgtgaatgttgacgacattgacttcttcttggttaactctttcgacgaactcgaagtcgaggtaattctcaactttagagttttgttgtttattgtatttttcttaattatctgcaaaatgcaatccaaataaaaatatttggaggtgatataattgtggatgatatatactactatcttactgaaccaattagtatatgatgtgaaataagtccttattccaatatagttgtagctagacttgtagctttctaggttcgtggtgaaatttttttaattcgatgttggtacataataatagcaaaaagatattagatagaaaacacatcattatttcaacggacaaaaagaacatcattcatcttggggccgaaagatatagatatatcgattggggcgaaaatcccaaggtgcagattacttataatatatcttatgcaattggttggggtcttaatttaacgtggcaaatatgtagccagaaaacgaatatcgtctcaaatatacatacgtgtcacgccgtttacctttccattgaaagcatacatactggctaggcatgtggggatgtgtccctgaccaaattgagattttgcacaacaatttgatttattttttaattctagtttcattagtttagttttatcctttattcttttggtttaattcattagtttatttcatatttttcttaacgatgtttgcattttcgcaaattactgtataaatgtgacatttgtttttaaaaatacttgctaaattgctttaatgcgtgtattaaagggaatgataccttttttattgtgtctttattacttccactggtccctattcatctaatgttcttgaagtcctttctaagcctgaaatcctgatattagaaaacgtggtcatgtttggttcaaactaggaattttagtccgaaagctttcacgataggtcgggagactcgggacggttgattgacacatttacatgtcaaagtgtctttttggggtatggccttatgggtttcagcttatctatcaatttcaaaatgaaacaggtttccttgttttaggaagaaaatagttgacgtgggtcatatactggtgacaaccaattattatagatgcaataaatgttatgattgtaattaatgaagtacataaaagcatgcatgaaattgatttgattgattttgaaactgatcttatatacaatcaacctatcaacgtttctttaatgcgtgtattaatccttttttcaggtgctacaatggatgaaaaaccaatggccggtcaagaacataggaccgatgattccatcaatgtacttagacaaacgattagcaggtgacaaagactacggaatcaacctcttcaatgcccaagtcaacgaatgccttgattggcttgactcaaaaccgcccggttcagtgatctacgtgtcttttggaagcttggccgtcttaaaagacgatcaaatgatagaagtcgcggctggtctaaaacaaactggccataacttcttatgggttgttagagaaactgaaacaaagaagcttccaagcaattacatagaggacatttgtgacaagggattgatagtgaattggagtcctcaattacaagttcttgcacataaatcaatcggttgtttcatgactcattgcgggtggaattcgactttagaggcattgagcttaggagttgctttgataggaatgccggcttatagcgaccagccgactaatgctaagtttattgaagatgtgtggaaggttggggttagggttaaggcagatcaaaatgggtttgttccgaaggaagagattgtgagatgtgttggagaagttatggaagatatgtcggagaaagggaaggagattagaaaaaatgctcggaggttgatggagtttgcaagggaagctttgtctgatggaggaaattctgataagaatattgatgagtttgttgctaaaattgtgaggtaa

SEQ ID NO: 2 - DNA, 1410 bp, Arabidopsis thaliana
atggcgccaccgcattttctactggtaacgtttccggcgcaaggtcacgtgaacccatctctccgttttgctcgtcggctcatcaaaagaaccggcgcacgtgtcactttcgtcacttgtgtctccgtcttccacaactccatgatcgcaaaccacaacaaagtcgaaaatctctctttccttactttctccgacggtttcgacgatggaggcatttccacctacgaagaccgtcagaaaaggtcggtgaatctcaaggttaacggcgataaggcactatcggatttcatcgaagctactaagaatggtgactctcccgtgacttgcttgatctacacgattcttctcaattgggctccaaaagtagcacgtagatttcaacttccctccgctcttctctggatccaaccggctttggttttcaacatctattacactcatttcatgggaaacaagtccgttttcgagttacctaatctgtcttctctggaaatcagagatcttccatctttcctcacaccttccaacacaaacaaaggcgcatacgatgcgtttcaagaaatgatggagtttctcataaaagaaaccaaaccgaaaattctcatcaacactttcgattcgctggaaccagaggccttaacggctttcccgaatatcgatatggtggcggttggtcctttacttcccacggagattttctcaggaagcaccaacaaatcagttaaagatcaaagtagtagttatacactttggctagactcgaaaacagagtcctctgttatttacgtttcctttggaacaatggttgagttgtccaagaaacagatagaggaactagcgagagcactcatagaagggaaacgaccgtttttgtgggttataactgataaatccaacagagaaacgaaaacagaaggagaagaagagacagagattgagaagatagctggattcagacacgagcttgaagaggttgggatgattgtgtcgtggtgttcgcagatagaggttttaagtcaccgagccgtaggttgttttgtgactcattgtgggtggagctcgacgctggagagtttggttcttggcgttccggttgtggcgtttccgatgtggtcggatcaaccgacgaacgcgaagctactggaagaaagttggaagactggtgtgagggtaagagagaacaaggatggtttggtggagagaggagagatcaggaggtgtttggaagccgtgatggaggagaagtcggtggagttgagggaaaacgcaaagaaatggaagcgtttagcgatggaagcgggtagagaaggaggatcttcggataagaacatggaggcttttgtggaggatatttgtggagaatctcttattcaaaacttgtgtgaagcagaggaggtaaaagtaaagtaa

SEQ ID NO: 3 - DNA, 1368 bp, Arabidopsis thaliana
atggcgcaaccgcattttctactggtaacgtttccggcgcaaggtcacgtgaacccatctctccgttttgctcgtcggctcatcaaaacaactggcgcacgtgtaactttcgccacgtgtctctctgtcattcaccgctctatgatcccaaaccacaacaacgtcgaaaatctctctttccttactttctccgacggattcgacgacggagtcatctccaacaccgacgacgtccaaaaccggttggtacacttcgaacgtaatggcgataaagctctatcggatttcatcgaagctaatcagaatggtgactctcccgtaagttgcttgatctacacgattcttcccaactgggttccaaaagtggcgcgtagatttcatcttccctctgttcatctctggatccaaccagccttcgctttcgacatttattacaattactctacaggaaacaactccgttttcgagttcccgaatctaccttctctcgaaatccgcgatctgccttctttcctctcaccttccaacacgaacaaagccgcacaagcagtatatcaagaactgatggattttctcaaagaagaatctaacccgaaaattctcgtcaacacattcgattcgctggagccagagttcttaacagctattccgaatatagaaatggtggcagttggtcctttacttcctgcggagattttcactggaagcgaatcaggtaaagatttatcaagagatcatcaaagtagtagttatacactttggttagactcgaaaacagagtcctctgttatttatgtttcttttggaacaatggttgagttgtcgaagaaacagatagaggaactagcgagagcactcatagaagggggaagaccgttcttgtgggttataactgataaactcaacagagaagcgaaaatagaaggagaagaagagacagagattgagaagatagctggttttagacacgagcttgaagaggttgggatgattgtctcgtggtgttcgcagatagaggttttgagacaccgagccataggttgttttttgactcattgtgggtggagctcatcactggagagtttggttctcggcgttccagtggtggcgtttccgatgtggtcggatcagccagcaaatgcgaagcttttggaagaaatatggaagacaggtgtgagggtgagagagaactcggaaggtttagtagagagaggagagataatgcggtgtttggaagcagtgatggaggcgaaatcggtggagctgagggaaaacgcagagaaatggaagcgtttagcgactgaagcgggtagagaaggaggatcttcggacaagaatgtggaagcttttgtgaagagtctgttttga

SEQ ID NO: 4 - DNA, 1371 bp, Arabidopsis thaliana
atgggcagtagtgagggtcaagaaacacatgtcctaatggtaacactaccattccaaggtcacatcaatccaatgctcaaactcgcaaaacatctctcgttatcatcaaagaacctacacatcaatctcgccactattgagtcagcccgtgatctcctctccaccgtagaaaaacctcgttatccggtggacctcgtgttcttctccgatggtctacctaaagaagatccaaaggcccctgaaactcttttgaagtcattgaataaagtcggagccatgaacttgtctaaaatcatcgaagaaaagagatactcttgtatcatctcttcgccttttactccatgggttccagctgttgcagcctctcataacatctcttgtgcaatactttggatccaagcttgtggagcttactcggtttattaccgttactacatgaagacaaactctttccctgatcttgaagatctgaatcaaacggtggagttaccagctttaccattgttggaagttcgagatcttccatcgtttatgttaccttctggtggtgctcacttctataatctaatggcggaatttgcagattgtttgaggtatgtgaaatgggttttggttaattcattctatgaactcgaatcagagataatcgaatcgatggctgatttaaaacctgtaattccaattggtcctctggtttctccatttctgttgggcgatggtgaggaggaaaccctagacggtaaaaacctagatttttgtaaatctgatgattgttgtatggagtggcttgacaagcaagctaggtcttctgttgtgtacatatctttcggaagtatgctcgaaacattggagaatcaggtcgagaccatagcgaaggcgctgaagaacagaggacttccatttctttgggtgataaggccaaaggagaaagcccaaaacgttgctgttttgcaggagatggtgaaagaaggacaaggggttgttctcgagtggagtccacaagagaagattttgagccacgaggcaatctcttgttttgtcacgcattgcggctggaactcgactatggagacggtggtggctggtgttcctgtggtagcgtaccctagctggacggatcagcccattgacgcgcggttgcttgttgatgtgtttggaatcggagtaaggatgaggaatgacagtgtcgatggcgagcttaaggtcgaagaagtagaaagatgcattgaggccgtgacggagggacccgctgccgtggatataagaaggagagcggcggagctaaagcgcgtggcgagattggcgttggcacctggtggatcttcgacacggaatttagacttgttcattagtgatatcacaatcgcctaa

SEQ ID NO: 5 - DNA, 1353 bp, Arabidopsis thaliana
atgggaagtaatgagggtcaagaaacacatgtcctaatggtagcattagcattccaaggtcatctcaatccaatgctcaaattcgcaaaacatctcgcacgaaccaatctacacttcactctcgccaccactgagcaagcccgtgacctcctctcttccaccgctgacgaacctcatagaccggtggacctcgctttcttctcagacggtctacctaaagacgatccaagagatcccgacactctcgcaaagtcattgaaaaaagatggagccaagaacttgtcaaaaatcatcgaagaaaagagatttgattgcatcatctctgtgccttttactccctgggttccagctgttgcagctgcacataacattccttgtgcaatcctctggatccaagcttgtggagctttttctgtttattaccgttattacatgaagacaaatcctttccccgaccttgaagatctgaatcaaacagtggagttaccagctttaccattgttggaagtccgagatctcccgtcattgatgttaccttctcaaggagctaatgtcaataccctaatggcggaatttgcagattgtttgaaagatgtgaaatgggttttggttaactcgttttacgaactcgaatcagagatcatcgagtctatgtctgatttaaaacctataatcccaattggtcctcttgtttctccattcctgttgggaaatgatgaagaaaaaaccctagatatgtggaaagttgatgattattgtatggagtggcttgacaagcaagctaggtcttcagttgtttacatatctttcggaagcatactcaaatcattggagaatcaagttgagaccatagcaacggcattaaaaaacagaggagttccatttctttgggtgatacggccgaaggagaaaggcgaaaacgtccaggttttgcaggagatggttaaagaaggtaaaggggttgtaactgaatggggtcaacaagaaaagatattgagccacatggcgatttcttgcttcatcacgcattgtggatggaactcgacgatcgagacggtggtgactggtgttcccgtggtggcgtatccgacttggatagatcagccgcttgatgcgagactgcttgtggatgtgtttggaatcggagtaaggatgaagaacgacgctatcgatggagagcttaaggttgcagaggtggagagatgcattgaggccgtgacagagggacctgccgccgcggatatgaggaggagagcgacggagctgaagcacgccgcaagatcggcgatgtcacctggtggatcttccgctcagaatttagactcgttcattagtgatatcccaatcacttga

SEQ ID NO: 6 - DNA, 1440 bp, Arabidopsis thaliana
atgaaaatagagctagtattcattccctctccggcaattagtcatctcatggcgacggtagagatggcggagcaactagttgataaaaacgacaacctctctatcaccgtaatcatcatatcttttagttctaaaaatacatccatgatcacctctcttacatccaacaaccgcctccggtacgaaataatctccggaggagatcaacaaccaacggagctcaaagcaactgattcccacatccaaagtctaaagccactggtgagagacgcggttgctaaactcgtagattccactctaccagacgcgcctcgtcttgcgggattcgttgttgacatgtactgcacgtcgatgatcgatgtcgctaacgaatttggcgtccctagttacttgttttacacctctaacgctggatttcttggacttttgcttcacattcagttcatgtacgatgcagaggatatctatgacatgagcgaattagaagactctgacgtagagttggtggttccgagtttgactagtccttatccgttgaaatgtcttccttacattttcaaatcaaaagagtggctcactttttttgtaactcaagcgagaagattcagagaaactaagggcattttggtaaacacggttcctgacttggaacctcaagcgttgacgtttctttccaatggtaacattccacgtgcttacccagtaggaccattgttgcatctcaaaaacgtaaattgtgattacgtggacaagaagcaatcggagattttacggtggctagacgagcaaccgccaagatctgtagtgttcctctgtttcgggagcatgggagggttcagtgaggaacaagtgagagaaaccgcattagctctcgatcgaagcggccaccggtttctttggtctctccgtcgtgcatctccgaatatattgagagagcctcccggagaattcacaaacctagaggagattctcccagaagggtttttcgatcggacggctaacagaggaaaggttatcggatgggctgaacaggtggccatattggcgaagccggcgatcggaggttttgtttctcacggcggatggaattcgacgttggagagtttgtggtttggtgttccgatggcgatttggccgctttacgctgaacagaagtttaacgctttcgagatggtggaagagcttggtttggctgtggagatcaagaagcattggcgaggagatcttttgttggggaggtcggagattgtgacggcggaggagattgagaaaggaatcatatgtttgatggagcaagacagtgacgtcaggaagagagtgaatgagatcagcgagaagtgccacgtggctttaatggacggtggatcgtcagaaactgctttgaaaagatttattcaagacgtaacggagaatattgcttggtcggaaactgaaagctag

(19) United States
(12) Patent Application Publication
(10) Pub. No.: US 2004/0241850 A1
(43) Pub. Date: Dec. 2, 2004

(54) Glucosyltransferases which glucosylate abscisic acid

(76) Inventors: Rosamond Jackson, University of York York (GB); Yi Li, University of York York (GB); Eng-Kiat Lim, University of York York (GB); Dianna Joy Bowles, University of York York (GB)

Correspondence Address: CROWELL & MORING LLP, INTELLECTUAL PROPERTY GROUP, P.O. BOX 14300, WASHINGTON, DC, 20044-4300, US

(21) Appl. No.: 10/489,254

(22) PCT Filed: Sep. 12, 2002
(86) PCT No.: PCT/GB02/04143

(30) Foreign Application Priority Data
Sep. 12, 2001 (GB) 0122110.0

(51) Int. Cl. A01H 1/00; C12N 15/82; C12N 5/04

(52) U.S. Cl. 435/419; 800/281

(57) ABSTRACT

The invention relates to transgenic cells which have been transformed with glucosyltransferase nucleic acids which encode glucosyltransferases which glucosylate abscisic acid, or analogues thereof; the use of said glucosyltransferases in screens for agents with herbicidal activity and in the production and/or testing of abscisic acid, or analogues thereof.

The invention relates to a glucosyltransferases which glucosylate abscisic acid, or analogues thereof, and the uses of said glucosyltransferases.

Glucosyltransferases (GTases) are enzymes which transfer glucosyl residues from activated nucleotide sugar to monomeric and polymeric acceptor molecules such as other sugars, proteins, lipids and other organic substrates. These glucosylated molecules take part in diverse metabolic pathways and processes. The transfer of a glucosyl moiety can alter the acceptors bioactivity, solubility and transport properties within the cell and throughout the plant. One family of GTases in higher plants is defined by the presence of a C-terminal consensus sequence. The GTases of this family function in the cytosol of plant cells and catalyse the transfer of glucose to small molecular weight substrates, such as phenylpropanoid derivatives, coumarins, flavonoids, other secondary metabolites and molecules known to act as plant hormones.

An example of a plant hormone, or phytohormone, is abscisic acid (ABA). ABA was first identified in the 1960's and shown to be responsible for the abscission of fruits. Two compounds were isolated and called abscisin I and abscisin II. Abscisin II is presently referred to as ABA. ABA is a naturally occurring compound in plants. It is a sesquiterpenoid which is partially produced by the mevalonic pathway in chloroplasts and other plastids. The production of ABA is stimulated by stresses such as water loss and freezing temperatures.

The physiological effects of ABA are varied. In contrast to other plant hormones, the endogenous concentrations of ABA can rise and fall dramatically in response to either environmental or development cues. For example, leaf ABA concentrations can increase 10-50 fold within a few hours of the onset of a water deficit. Subsequently re-watering will return the concentrations to normal over the same time period. As mentioned above ABA is involved in a variety of physiological processes, including by example, embryo development, seed dormancy, transpiration and adaptation to environmental stresses. ABA regulates many agronomically important aspects of plant development including synthesis of seed storage proteins and lipids as well as regulating stomatal closure.

By controlling the expression levels of an ABA GTase in plants (knocking out, overexpressing or more specific modulation using inducible/developmental promoters) the levels of free ABA can by regulated. This has clear utility in, for example, controlling germination timing or drought tolerance.

ABA inhibits seed germination preventing seed sprouting. Once ABA levels drop below a certain threshold germination occurs. Light rain can trigger germination too early in the growing season but if ABA GTase is downregulated the ABA level may remain high for longer and so delay germination which is beneficial if it allows a plant to delay germination until better growth conditions occur.

ABA has a major function in maintaining water balance as it induces the closure of the stomata during water shortage. Modulation of ABA levels would enable the production of plants with a greater drought tolerance by controlling the signal transduction pathway leading to stomatal opening.

The involvement of glucosylation in the bioactivity of ABA is controversial. Glucose conjugates of ABA have little or no biological activity and are not considered to be a reserve or storage form of ABA. In some tissues, the formation of ABA-glucose ester or other conjugates appears to be a major pathway for the inactivation of ABA.

Mutations in ABA synthesis are known in a variety of plant species, see Leung and Giraudat (1998) Annual Review of Plant Physiol. Plant Mol. Biol. In Arabidopsis thaliana a number of mutants have been identified which were selected based on the ability of the seeds to germinate in the presence of inhibitory concentrations of ABA. The mutations have also been shown to affect several additional aspects of seed development, including accumulation of storage proteins and lipids, chlorophyll breakdown and desiccation tolerance. In addition five mutationally identified ABA response loci have been cloned. These represent three classes of proteins. The classes include two orthologous transcriptional regulators (viviparous 1— Vp1) of maize and ABA—insensitive-3 of Arabidopsis (ABI3), two highly homologous members of the protein phosphatase 2 C family, and a farnesyl transferase of Arabidopsis, see McCartyet al (1991) Cell, 66: 895-905; Giraudat et al (1992) Plant Cell 4:1251-1261; Leung et al (1994) Science 264: 1448-1452; Cuither et al (1996) Science, 273:1239-1241.

We have identified a plant GTase, referred to as UGT71B6, which glucosylates ABA which has utility with respect to many aspects of plant biochemistry and physiology. For example, to modulate the levels of ABA in planta; in screening methods to identify agents with herbicidal activity; in screening methods to identify ABA analogues with biological activity which are not glucosylated or show reduced glucosylation; and the use of ABA glucosyltransferases in biotransformation to select for particular forms of ABA.

According to an aspect of the invention there is provided a transgenic cell comprising a nucleic acid molecule which comprises a nucleic acid sequence which encodes a polypeptide wherein said nucleic acid molecule is selected from the group consisting of:

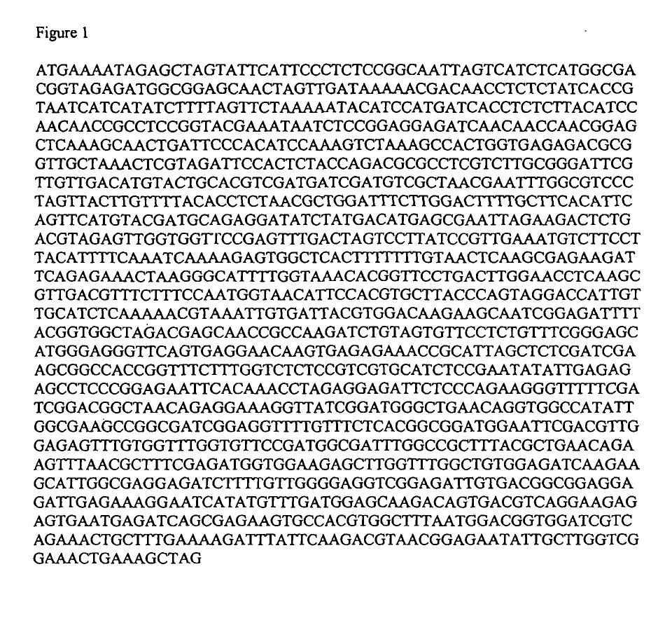

i) nucleic acid molecules consisting of the sequences as represented in FIGS. 1-6;

ii) nucleic acid molecule which hybridise to the sequences of (i) above and which glucosylate abscisic acid, or analogue thereof; and

iii) nucleic acid molecules consisting of sequences which are degenerate as a result of the genetic code to the sequences defined in (i) and (ii) above.

In a preferred embodiment of the invention said nucleic acid molecule hybridises under stringent hybridisation conditions to the sequences represented FIGS. 1-6.

Stringent hybridisation/washing conditions are well known in the art. For example, nucleic acid hybrids that are stable after washing in 0.1×SSC, 0.1% SDS at 60° C. It is well known in the art that optimal hybridisation conditions can be calculated if the sequence of the nucleic acid is known. For example, hybridisation conditions can be determined by the GC content of the nucleic acid subject to hybridisation. Please see Sambrook et al (1989) Molecular Cloning; A Laboratory Approach. A common formula for calculating the stringency conditions required to achieve hybridisation between nucleic acid molecules of a specified homology is:

Tₘ=81.5° C.+16.6 Log [Na⁺]+0.41[% G+C]−0.63(% formamide).

Typically, hybridisation conditions uses 4-6×SSPE (20×SSPE contains 175.3 g NaCl, 88.2 g NaH₂PO₄H₂O and 7.4 g EDTA dissolved to 1 litre and the pH adjusted to 7.4); 5-10× Denhardts solution (50× Denhardts solution contains 5 g Ficoll (type 400, Pharmacia), 5 g polyvinylpyrrolidone abd 5 g bovine serum albumen; 100 μg-1.0 mg/ml sonicated salmon/herring DNA; 0.1-1.0% sodium dodecyl sulphate; optionally 40-60% deionised formamide. Hybridisation temperature will vary depending on the GC content of the nucleic acid target sequence but will typically be between 42°-65° C.

In a preferred embodiment of the invention said transgenic cell over-expresses said abscisic acid glucosyltransferase.

In a preferred embodiment of the invention said over-expression is at least 2-fold higher when compared to a non-transformed reference cell of the same species. Preferably said over-expression is: at least 3-fold, 4-fold, 5-fold, 6-fold, 7-fold, 8-fold, 9-fold, or at least 10-fold when compared to a non-transformed reference cell of the same species.

It will be apparent that over-expression of an ABA glucosyltransferase can be achieved by providing a transgenic cell with multiple copies of a nucleic acid molecule encoding said glucosyltransferase or by placing the expression of said glucosyltransferase under the control of a strong constitutive or inducible promoter.

In an alternative preferred embodiment of the invention there is provided a transgenic cell wherein the genome of said cell is modified such that the activity of said abscisic acid glucosyltransferase is reduced when compared to a non-transgenic reference cell of the same species.

In a preferred embodiment of the invention said activity is reduced by at least 10%. Preferably said activity is reduced by between 10%-99%. Preferably said activity is reduced by at least 20%, 30%, 40%, 50%, 60%, 70%, 80%, or at least 90% when compared to a non-transgenic reference cell.

In a preferred embodiment of the invention said nucleic acid molecule is a cDNA.

In yet a further preferred embodiment of the invention said nucleic acid molecule is a genomic DNA.

In a preferred embodiment of the invention said transgenic cell is a eukaryotic cell. Preferably a mammalian cell, for example a human cell.

In a further preferred embodiment of the invention said eukaryotic cell is a plant cell.

Plants which include a plant cell according to the invention are also provided as are seeds produced by said plants.

In a preferred embodiment of the invention said plant is selected from: corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (helianthus annuas), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassaya (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plants of the present invention are crop plants (for example, cereals and pulses, maize, wheat, potatoes, tapioca, rice, sorghum, millet, cassava, barley, pea, and other root, tuber or seed crops. Important seed crops are oil-seed rape, sugar beet, maize, sunflower, soybean, and sorghum. Horticultural plants to which the present invention may be applied may include lettuce, endive, and vegetable brassicas including cabbage, broccoli, and cauliflower, and carnations and geraniums. The present invention may be applied in tobacco, cucurbits, carrot, strawberry, sunflower, tomato, pepper, chrysanthemum.

Grain plants that provide seeds of interest include oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava been, lentils, chickpea.

In a further preferred embodiment of the invention said plant is selected from the following group: maize; tobacco; oil seed rape; potato; soybean.

In a further preferred embodiment of the invention said eukaryotic cell is a fungal cell, preferably a yeast cell. More preferably still said yeast cell is selected from the following list: Saccharomyces spp eg Saccharomyces cerevisiae; Pichia spp.

In a further preferred embodiment of the invention said transgenic cell is null for a nucleic acid sequence selected from the group consisting of:

i) a nucleic acid sequence as represented in FIGS. 1-6;

ii) nucleic acid sequences which hybridise to the sequences of (i) above and which have glucosylate abscisic acid; and

iii) nucleic acid sequences which are degenerate as a result of the genetic code to the sequences defined in (i) and (ii) above.

Null refers to a cell which includes a non-functional copy of the nucleic acid sequence described above. Methods to provide such a cell are well known in the art and include the use of antisense genes to regulate the expression of specific targets; insertional mutagenesis using T-DNA; and double stranded inhibitory RNA (RNAi).

According to a further aspect of the invention there is provided an antisense sequence, or part thereof, of the sense sequence represented in FIGS. 1-6.

Preferably said antisense sequence is derived from the 3′ untranslated region of the sense sequences represented in FIGS. 1-6. More preferably the antisense sequence is at least 50 base pairs 3′ to the termination codon. More preferably still said antisense sequence is 100-300 base pairs 3′ to the termination codon.

According to a further aspect of the invention there is provided a vector comprising a nucleic acid molecule selected from the following group:

i) nucleic acid molecules consisting of sequences represented in FIGS. 1-6;

ii) nucleic acid molecules which hybridise to the sequences represented in (i) and which glucosylate abscisic acid, or an analogues thereof; and

iii) nucleic acid molecules consisting of sequences which are degenerate as a result of the genetic code to sequences defined in (ii) and (iii) above.

In a preferred embodiment of the invention said nucleic acid molecule is the antisense sequence of the sequence represented by (i), (ii) or (iii) above.

Suitable vectors can be constructed, containing appropriate regulatory sequences, including promoter sequences, terminator fragments, polyadenylation sequences, enhancer sequences, marker genes and other sequences as appropriate. For further details see, for example, Molecular Cloning: Laboratory Manual: 2^{nd} edition, Sambrook et al. 1989, Cold Spring Habor Laboratory Press or Current Protocols in Molecular Biology, Second Edition, Ausubel et al. Eds., John Wiley & Sons, 1992.

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, (e.g. bacterial), or plant cell. The vector may be a bi-functional expression vector which functions in multiple hosts. In the case of GTase genomic DNA this may contain its own promoter or other regulatory elements and in the case of cDNA this may be under the control of an appropriate promoter or other regulatory elements for expression in the host cell.

By “promoter” is meant a nucleotide sequence upstream from the transcriptional initiation site and which contains all the regulatory regions required for transcription. Suitable promoters include constitutive, tissue-specific, inducible, developmental or other promoters for expression in plant cells comprised in plants depending on design. Such promoters include viral, fungal, bacterial, animal and plant-derived promoters capable of functioning in plant cells.

Constitutive promoters include, for example CaMV 35S promoter (Odell et al. (1985) Nature 313, 9810-812); rice actin (McElroy et al. (1990) Plant Cell 2: 163-171); ubiquitin (Christian et al. (1989) Plant Mol. Biol. 18 (675-689); pEMU (Last et al. (1991) Theor Appl. Genet. 81: 581-588); MAS (Velten et al. (1984) EMBO J. 3. 2723-2730); ALS promoter (U.S. Application Seriel No. 08/409,297), and the like. Other constitutive promoters include those in U.S. Pat. Nos. 5,608,149; 5,608,144; 5,604,121; 5,569,597; 5,466,785; 5,399,680, 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induced gene expression, or a chemical-repressible promoter, where application of the chemical represses gene expression. Chemical-inducible promoters are known in the art and include, but are not limited to, the maize 112-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88: 10421-10425 and McNellis et al. (1998) Plant J. 14(2): 247-257) and tetracycline-inducible and tetracycline-repressible promoters (see, for example, Gatz et al. (1991) Mol. Gen. Genet. 227: 229-237, and U.S. Pat. Nos. 5,814,618 and 5,789,156, herein incorporated by reference.

Where enhanced expression in particular tissues is desired, tissue-specific promoters can be utilised. Tissue-specific promoters include those described by Yamamoto et al. (1997) Plant J. 12(2): 255-265; Kawamata et al. (1997) Plant Cell Physiol. 38(7): 792-803; Hansen et al. (1997) Mol. Gen. Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascni et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol. Biol. 23(6): 1129-1138; Mutsuoka et al. (1993) Proc. Natl. Acad. Sci. USA 90 (20): 9586-9590; and Guevara-Garcia et al (1993) Plant J. 4(3): 495-50.

“Operably linked” means joined as part of the same nucleic acid molecule, suitably positioned and oriented for transcription to be initiated from the promoter. DNA operably linked to a promoter is “under transcriptional initiation regulation” of the promoter. In a preferred aspect, the promoter is an inducible promoter or a developmentally regulated promoter.

Particular of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148. Suitable vectors may include plant viral-derived vectors (see e.g. EP-A-194809).

If desired, selectable genetic markers may be included in the construct, such as those that confer selectable phenotypes such as resistance to antibodies or herbicides (e.g. kanamycin, hygromycin, phosphinotricin, chlorsulfuron, methotrexate, gentamycin, spectinomycin, imidazolinones and glyphosate).

Plants transformed with a DNA construct of the invention may be produced by standard techniques known in the art for the genetic manipulation of plants. DNA can be introduced into plant cells using any suitable technology, such as a disarmed Ti-plasmid vector carried by Agrobacterium exploiting its natural gene transferability (EP-A-270355, EP-A-0116718, NAR 12(22):8711-87215 (1984), Townsend et al., U.S. Pat. No. 5,563,055); particle or microprojectile bombardment (U.S. Pat. No. 5,100,792, EP-A-444882, EP-A-434616; Sanford et al, U.S. Pat. No. 4,945,050; Tomes et al. (1995) “Direct DNA Transfer into Intact Plant Cells via Microprojectile Bombardment”, in Plant Cell, Tissue and Organ Culture: Fundamental Methods, ed. Gamborg and Phillips (Springer-Verlag, Berlin); and McCabe et al. (1988) Biotechnology 6: 923-926); microinjection (WO 92/09696, WO 94/00583, EP 331083, EP 175966, Green et al. 91987) Plant Tissue and Cell Culture, Academic Press, Crossway et al. (1986) Biotechniques 4:320-334); electroporation (EP 290395, WO 8706614, Riggs et al. (1986) Proc. Natl. Acad. Sci. USA 83:5602-5606; D'Halluin et al. 91992). Plant Cell 4:1495-1505) other forms of direct DNA uptake (DE 4005152, WO 9012096, U.S. Pat. No. 4,684,611, Paszkowski et al. (1984) EMBO J. 3:2717-2722); liposome-mediated DNA uptake (e.g. Freeman et al (1984) Plant Cell Physiol, 29:1353); or the vortexing method (e.g. Kindle (1990) Proc. Nat. Acad. Sci. USA 87:1228). Physical methods for the transformation of plant cells are reviewed in Oard (1991) Biotech. Adv. 9:1-11. See generally, Weissinger et al. (1988) Ann. Rev. Genet. 22:421-477; Sanford et al. (1987) Particulate Sciences and Technology 5:27-37; Christou et al. (1988) Plant Physiol. 87:671-674; McCabe et al. (1988) Bio/Technology 6:923-926; Finer and McMullen (1991) In Vitro Cell Dev. Biol. 27P:175-182; Singh et al. (1988) Theor. Appl. Genet. 96:319-324; Datta et al. (1990) Biotechnology 8:736-740; Klein et al. (1988) Proc. Natl. Acad. Sci. USA 85: 4305-4309; Klein et al. (1988) Biotechnology 6:559-563; Tomes, U.S. Pat. No. 5,240,855; Buising et al. U.S. Pat. Nos. 5,322,783 and 5,324,646; Klein et al. (1988) Plant Physiol 91: 440-444; Fromm et al (1990) Biotechnology 8:833-839; Hooykaas-Von Slogteren et al. 91984). Nature (London) 311:763-764; Bytebier et al. (1987) Proc. Natl. Acad. Sci. USA 84:5345-5349; De Wet et al. (1985) in The Experimental Manipuation of Ovule Tissues ed. Chapman et al. (Longman, N.Y.), pp. 197-209; Kaeppler et al. (1990) Plant Cell Reports 9:415-418 and Kaeppler et al. (1992) Theor. Appl. Genet. 84:560-566; Li et al. (1993) Plant Cell Reports 12: 250-255 and Christou and Ford (1995) Annals of Botany 75: 407-413; Osjoda et al. (1996) Nature Biotechnology 14:745-750, all of which are herein incorporated by reference.

Agrobacterium transformation is widely used by those skilled in the art to transform dicotyledonous species. Recently, there has been substantial progress towards the routine production of stable, fertile transgenic plants in almost all economically relevant monocot plants (Toriyama et al. (1988) Bio/Technology 6: 1072-1074; Zhang et al. (1988) Plant Cell rep. 7379-384; Zhang et al. (1988) Theor. Appl. Genet. 76:835-840; Shimamoto et al. (1989) Nature 338:274-276; Datta et al. (1990) Bio/Technology 8: 736-740; Christou et al. (1991) Bio/Technology 9:957-962; Peng et al (1991) International Rice Research Institute, Manila, Philippines, pp.563-574; Cao et al. (1992) Plant Cell Rep. 11: 585-591; Li et al. (1993) Plant Cell Rep. 12: 250-255; Rathore et al. (1993) Plant Mol. Biol. 21:871-884; Fromm et al (1990) Bio/Technology 8:833-839; Gordon Kamm et al. (1990) Plant Cell 2:603-618; D'Halluin et al. (1992) Plant Cell 4:1495-1505; Walters et al. (1992) Plant Mol. Biol. 18:189-200; Koziel et al. (1993). Biotechnology 11194-200; Vasil, I. K. (1994) Plant Mol. Biol. 25:925-937; Weeks et al (1993) Plant Physiol. 102:1077-1084; Somers et al. (1992) Bio/Technology 10:1589-1594; WO 92/14828. In particular, Agrobacterium mediated transformation is now emerging also as an highly efficient transformation method in monocots. (Hici, et al. (1994) The Plant Journal 6:271-282). See also, Shimamoto, K. (1994) Current Opinion in Biotechnology 5:158-162; Vasil, et al. (1992) Bio/Technology 10:667-674; Vain, et al. (1995) Biotechnology Advances 13(4):653-671; Vasil, et al. (1996) Nature Biotechnology 14: 702).

Microprojectile bombardment, electroporation and direct DNA uptake are preferred where Agrobacterium is inefficient or ineffective. Alternatively, a combination of different techniques may be employed to enhance the efficiency of the transformation process, e.g. bombardment with Agrobacterium-coated microparticles (EP-A-486234) or microprojectile bombardment to induce wounding followed by co-cultivation with Agrobacterium (EP-A-486233).

According to a further aspect of the invention there is provided a method for the production of glucosylated abscisic acid, or derivatives or analogue thereof, comprising:

i) culturing a transgenic cell according to the invention;

ii) providing conditions which facilitate the production of glucosylated abscisic acid by said cell; and optionally

iii) isolating the glucosylated abscisic acid from the cell or the cell-culture medium.

In a preferred method of the invention said glucosylated abscisic acid is the (+) abscisic acid enantiomer.

In an alternative method of the invention said glucosylated abscisic acid is the (−) abscisic acid enantiomer.

In a preferred method of the invention said cell is a eukaryotic cell. Preferably said cell is a fungal cell.

In an alternative preferred method of the invention said cell is a prokaryotic cell.

According to a further aspect of the invention there is provided a screening method for the identification an agent with the ability to inhibit plant growth and/or viability comprising the steps of:

i) providing a polypeptide encoded by a nucleic acid molecule selected from the following group;

a) a nucleic acid molecule consisting of a nucleic acid sequence represented in FIGS. 1-6;

b) nucleic acid molecules which hybridise to the sequences of (i) above and which have glucosyltransferase activity; and

c) nucleic acid molecules consisting of sequences which are degenerate as a result of the genetic code to the sequences defined in (a) and (b) above;

ii) providing at least one candidate agent;

iii) forming a preparation of (i) and (ii);

iv) providing a detectable amount of abscisic acid;

v) detecting or measuring the glucosylation activity of the polypeptide in (i) with respect to abscisic acid in (iv); and optionally

vi) testing the effect of the agent on the growth and/or viability of plants.

In a preferred method of the invention said agent has herbicidal activity.

In a preferred method of the invention said polypeptide is encoded the nucleic acid molecule consisting of a nucleic acid sequence represented in FIGS. 1-6.

In a further preferred method of the invention abscisic acid is provided at bewteen about 0.1 mM and 2.0 mM ABA. Preferably about 1 mM ABA.

In a further preferred method according to the invention said polypeptide in (i) is recombinantly manufactured.

In an alternative preferred method said polypeptide is expressed by a cell according to the invention and the preparation in (iii) is a cell in culture and said agent is added to said cell culture.

Preferably said cell is selected from the following group: plant cell; fungal cell; bacterial cell; mammalian cell.

According to a further aspect of the invention there is provided an agent identified by the method according to the invention.

In a preferred embodiment of the invention said agent is combined with a carrier typically used in herbicidal compositions.

According to a further aspect of the invention there is provided a method to test a herbicidal agent for inhibitory activity with respect to glucosylation of abscisic acid comprising:

i) providing a transgenic plant or plant cell according to the invention;

ii) applying an agent to be tested to said plant or plant cell;

iii) detecting or measuring the effect of the agent on said plant or plant cell growth and/or viability;

iv) comparing the growth and/or viability of the treated plants or plant cells with an untreated control plant or plant cell; and optionally

v) applying the agent to a non-transgenic plant or plant cell to test for efficacy.

According to a further aspect of the invention there is provided a method for inhibiting the growth of undesired vegetation comprising applying an agent identified by the methods according to the invention.

According to a further aspect of the invention there is provided a polypeptide encoded by a nucleic acid molecule selected from the group consisting of:

i) a nucleic acid molecule consisting of a nucleic acid sequence as represented in FIGS. 1-6;

ii) nucleic acid molecules which hybridise to the sequences of (i) above and which glucosylate abscisic acid; and

iii) nucleic acid molecules consisting of nucleic acid sequences which are degenerate as a result of the genetic code to the sequences defined in (i) and (ii) above

for use in the in vitro modification of abscisic acid, or analogues thereof.

According to a further aspect of the invention there is provided a method to test the activity of an abscisic acid glucosyltransferase to modify an abscisic acid analogue comprising the steps of:

i) forming a preparation of an abscisic acid glucosyltransferase and at least one abscisic acid analogue; and

ii) determining the presence, or not, of a glucosyl moiety conjugated to said abscisic acid analogue.

It is desirable to identify abscisic acid analogues which retain biological activity but are not glucosylated. Glucosylation of abscisic acid in planta results in inactivation of abscisic acid and ablation of biological activity. This severely restricts the use of abscisic acid as an agrochemical. The ability to screen analogues of abscisic acid with abscisic acid glucosyltransferases is valuable because it allows analogues with abscisic acid activity to be tested prior to field studies. Analogues of abscisic acid are known in the art, for example, U.S. Pat. No. 5,481,034, which is incorporated by reference.

Moreover, a second obstacle to the use of abscisic acid as an agrochemical agent is the presence of 7′ and 8′ hydroxylases in planta which inactivate abscisic acid by hydroxylation. It is known that abscisic acid analogues which are not hydroxlated by 8′-hydroxylation are long lived, (see Abrams et al Plant Physiol. 114:89-97, which is incorporated by reference). It would therefore be desirable to also test analogues, which have initially been screened for glucosylation, for the lack of hydroxylation by 7′ and 8′ hydroxylase. Plants which are exposed to long-lived abscisic acid analogues have several desirable characteristics, for example, enhanced oil accumulation in oil seeds, dessication tolerance and delayed germination.

In a preferred method of the invention said analogue is tested for resistance to 7′ and/or 8′ hydroxylation.

7′ and 8′ hydroxylases are known in the art. For example, see WO0246377, which is incorporated by reference.

According to a further aspect of the invention there is provided an in vitro method for the production of glucosylated abscisic acid comprising the steps of:

i) providing a preparation of an abscisic acid glucosyltransferase and abscisic acid; and

ii) providing reaction conditions which facilitate the addition of at least one glucosyl moiety to abscisic acid.

In a preferred method of the invention said glucosylated abscisic acid is the (+) abscisic acid enantiomer.

In an alternative method of the invention said glucosylated abscisic acid is the (−) abscisic acid enantiomer.

According to a yet further aspect of the invention there is provided a method for the preparation of (+) abscisic acid enantiomer from a racemic mixture of abscisic acid comprising the steps of:

i) forming a preparation of at least one abscisic acid glucosyltransferase and a racemic mixture of abscisic acid;

ii) providing reaction conditions which facilitate the formation of a (+) abscisic acid enantiomer from said racemic mixture.

An embodiment of the invention will now be described by example only and with reference to the following figures,

FIG. 1 represents the nucleic acid sequence of 71B6;

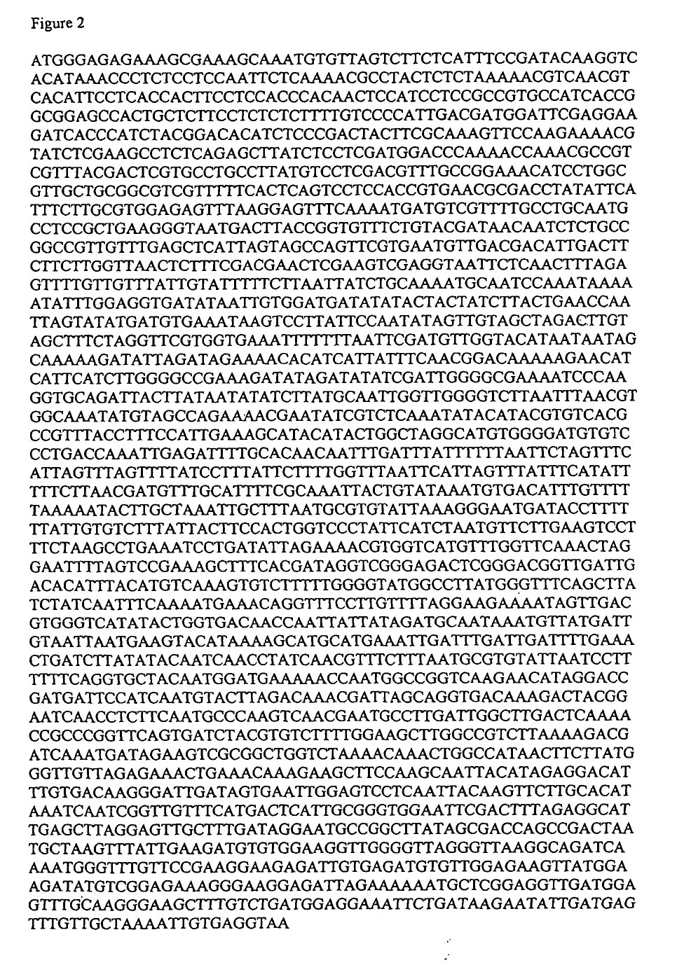

FIG. 2 represents the nucleic acid sequence of 74D1;

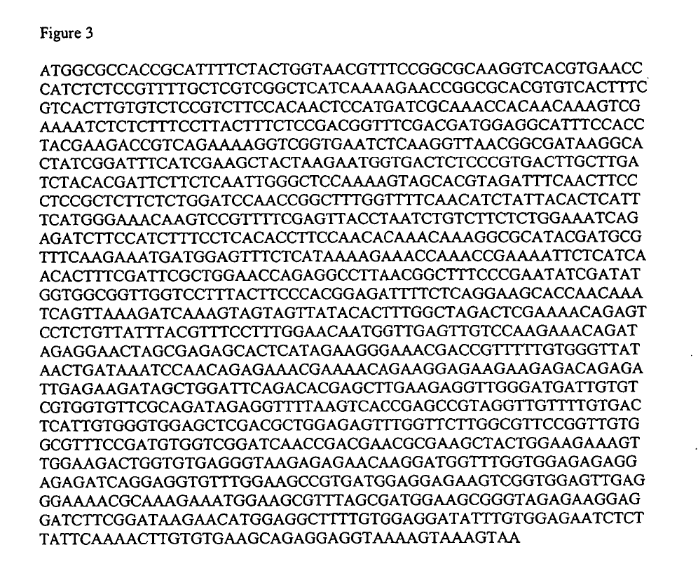

FIG. 3 represents the nucleic acid sequence of 75B1;

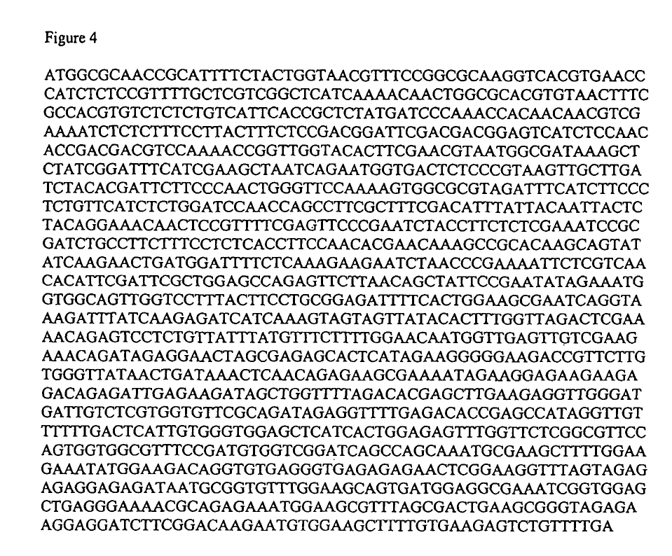

FIG. 4 represents the nucleic acid sequence of 75B2;

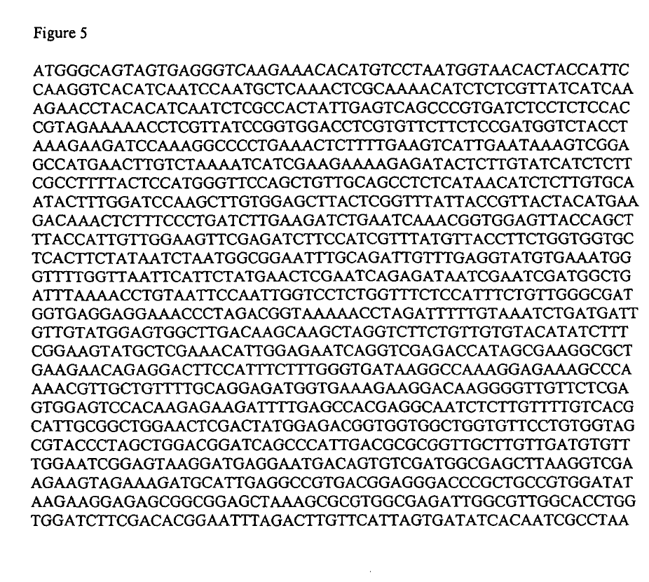

FIG. 5 represents the nucleic acid sequence of 84B1;

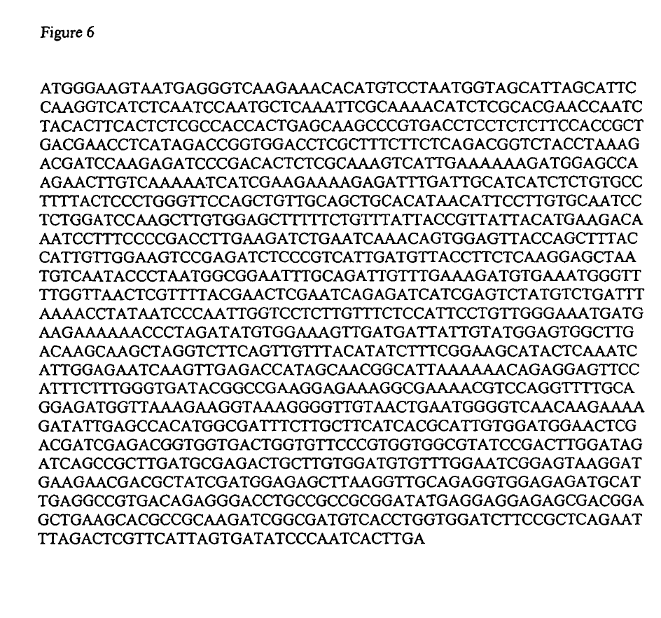

FIG. 6 represents the nucleic acid sequence of 84B2; and

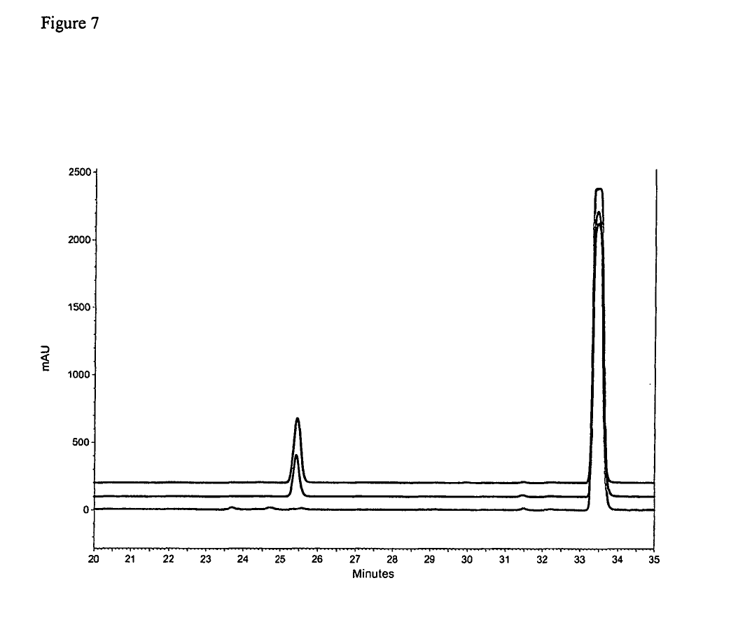

FIG. 7 is a HPLC scan of ABA glucosylated in vitro by 71B6 (bottom trace) and 0.84B1 (top trace);

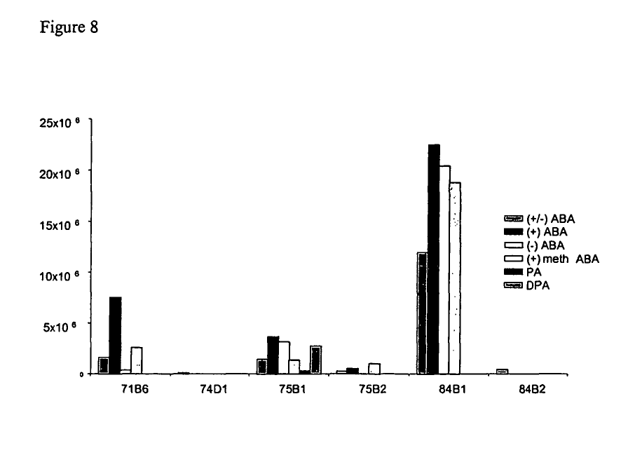

FIG. 8 illustrates the relative activity of UGTs 71B6, 74D1, 75B1, 75B2, 84B1 and 84B2 towards ABA and related substrates. All assays were carried out in 50 mM TRIS pH 7.0, 14 mM 2-mercaptoethanol, 0.5 mM substrate, 5 mM UDPG and 10 μg/ml enzyme. The reactions were incubated at 30° C. for 30 min; and

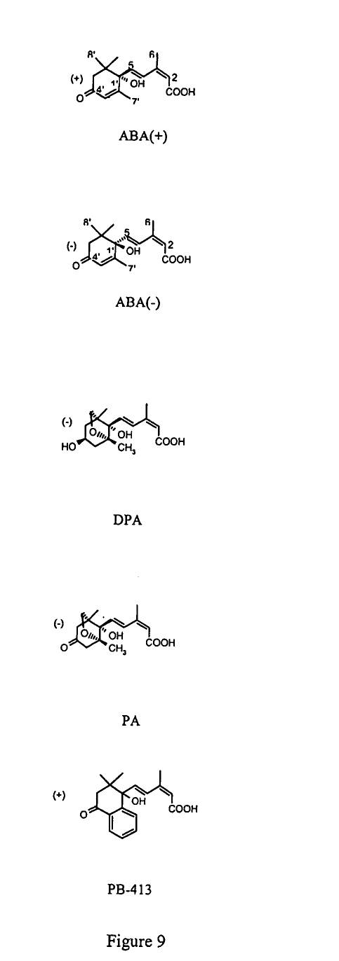

FIG. 9 illustrates the chemical structure of (+) and (−) abscisic acid enantiomers and examples of abscisic acid analogues.

Materials and Methods

Plant Materials

Wild-type Arabidopsis, ecotype Columbia, were grown in Levingtons seed and modular compost in a controlled environment of 16 h/8 h light-dark cycle (22° C., 170 μEm⁻²s⁻¹ light, 18° C., dark).

Recombinant UGT Purification

Escherichia coli strain XL-1 Blue carrying the recombinant GST-UGT protein expression plasmid *(27) was grown at 20° C. in 75 ml 2×YT media containing 50 μg/ml ampicillin until the A₆₀₀ nm reached 1.0, after which the culture was incubated with 1 mM isopropyl-1-thio-β-D-galactopyranoside for 24 h at 20° C. The cells were harvested by centrifugation at 5,000×g for 5 min and were resuspended in 2 ml of Spheroblast buffer (0.5 mM EDTA, 750 mM sucrose, 200 mM Tris-HCl, pH 8.0) *(28). Lysozyme (1 mg) and 14 ml of half-strength Spheroblast buffer were added immediately. After incubation at 4° C. for 30 min, the cells were harvested again by centrifugation, and osmotically shocked in 5 ml of phosphate-buffered saline containing 0.2 mM phenylmethylsulphonylfluoride. Cell debris was removed by centrifugation at 10,000×g for 15 min. The protein in the supernatant fraction was collected by adding 100 μl of 50% glutathione-coupled sepharose gel (Pharmacia), and recovered in elution buffer (20 mM reduced-form glutathione, 100 mM Tris-HCl, pH 8.0, 120 mM NaCl), according to the manufacturer's instructions. The protein assays were carried out with Bio-Rad Protein Assay Dye using bovine serum albumin as reference. The purified recombinant proteins were also analysed by SDS-PAGE following the methods described by Sambrook et al. *(29).

Glucosyltransferase Activity Assay

The general glucosyltransferase activity assay mix (200 μl) contained 2 μg of purified recombinant proteins, 14 mM 2-mercaptoehanol, 2.5 mM UDPG, 1 mM ABA, 50 mM Tris-HCl, pH 7.0. The reaction was carried out at 30° C. for 1 h, and stopped by the addition of 20 μl TCA (240 mg/ml). The reaction mix was analysed using the HPLC method.

HPLC Analysis

Reverse phase HPLC was performed with Waters HPLC System (Waters Separator 2690 and Waters Tunable Absorbance Detector 486, Waters Limited, Herts, UK) and a Columbus 5μ C₁₈ column (250×4.60 mm, Phenomenex). A linear gradient with increasing methanol (solvent B) against distilled H₂O (solvent A) at a flow rate 1 ml/min over 40 min was used to separate the glucose conjugate from their aglycone. Both solvents contained 0.01% H₃PO₄ (pH 3.0). The following elution conditions were used: ABA, 10-70% B, λ_{detection} 275 nm.

Coupled Enzyme Assay

The ABA-UGT activity was determined as the release of UDP, which can be measured using a coupled assay containing UGT, pyruvate kinase and lactate dehydrogenease (30). The reaction mechanisms are shown as the following:

ABA+UDPG [symbol] ABA−Glc+UDP

PEP+UDP [symbol] UTP+pyruvate

Pyruvate+NADH+H⁺ [symbol] lactate+NAD⁺

The reaction mix, in a total volume of 1.0 ml, contained 50 mM HEPES-NaOH pH 7.6, 2.5 mM MgSO₄, 10 mM KCl, 0.15 mM NADH, 2.0 mM phosphoenol pyruvate (PEP), 10 μl of UGT solution (diluted into 50 mM HEPES-NaOH pH 7.6), 3.0 units of pyruvate kinase and 4.0 units of lactate dehydrogenase. The coupled enzyme assay was analysed over the range 0-5 mM UDPG and 0-1 mM ABA together with a control at the same concentration of UDPG but with no ABA nor UGT. The change of NAD⁺ was detected at 340 nm, and the reaction rate was converted to the unit mkat kg⁻¹ using the extinction coefficient 6.22×10³ M⁻¹ cm⁻¹ for NADH.

Analysis of Abscisic Acid Enantiomers

The analysis of reaction products of racemic mixtures of abscisic acid or analogues thereof with glucosyltransferases is performed by methods well known in the art. Reaction products are typically analysed on a chiral HLPC column which resolves enantiomers of abscisic acid.

Examples of the separation of abscisic acid by chiral HPLC can be found at www.registech.com/chiral/applications/ or www.chromtech.se/chiral.htm.

1-35. (canceled).

36. A transgenic plant cell comprising a nucleic acid molecule which comprises a nucleic acid sequence selected from the group consisting of SEQ ID NO:1, SEQ ID NO:2, SEQ ID NO:3, SEQ ID NO:4, SEQ ID NO:5, SEQ ID NO:6 and nucleic acid sequences which hybridise to any of the foregoing sequences under stringent hybridization conditions, wherein said nucleic acid molecule encodes a glucosyltransferase polypeptide which glucosylates abscisic acid or an analogue of abscisic acid.

37. A cell according to claim 36, wherein said cell over-expresses the abscisic acid glucosyltransferase.

38. A cell according to claim 37, wherein the cell over-expresses the abscisic acid glucosyltransferase by at least two-fold when compared to a non-transgenic reference cell of the same species.

39. A cell according to claim 37, wherein the cell over-expresses the abscisic acid glucosyltransferase by at least 4-fold, when compared to a non-transformed reference cell of the same species.

40. A cell according to claim 39, wherein the cell over-expresses the abscisic acid glucosyltransferase by at least 7-fold, when compared to a non-transformed reference cell of the same species.

41. A cell according to claim 40, wherein the cell over-expresses the abscisic acid glucosyltransferase by at least 10-fold, when compared to a non-transformed reference cell of the same species.

42. A cell according to claim 36, wherein the genome of the cell is modified such that the activity of the abscisic acid glucosyltransferase is reduced when compared to a non-transgenic reference cell of the same species.

43. A cell according to claim 42, wherein the abscisic acid glucosyltransferase activity is reduced by at least 10%.

44. A cell according to claim 43, wherein the abscisic acid glucosyltransferase activity is reduced by at least 30% when compared to a non-transgenic reference cell.

45. A cell according to claim 44, wherein the abscisic acid glucosyltransferase activity is reduced by at least 60% when compared to a non-transgenic reference cell.

46. A cell according to claim 45, wherein the abscisic acid glucosyltransferase activity is reduced by at least 90% when compared to a non-transgenic reference cell.

47. A cell according to claim 36, wherein said nucleic acid molecule is a cDNA.

48. A cell according to claim 36, wherein said nucleic acid molecule is a genomic DNA.

49. A transgenic plant comprising a cell according to claim 36.

50. A transgenic seed comprising a cell according to claim 36.

51. A cell according to claim 36, wherein said cell is null for said nucleic acid molecule.

52. A method for producing glucosylated abscisic acid or a derivative or analogue thereof, comprising:
i) culturing a transgenic cell comprising a nucleic acid molecule which comprises a nucleic acid sequence selected from the group consisting of SEQ ID NO:1, SEQ ID NO:2, SEQ ID NO:3, SEQ ID NO:4, SEQ ID NO:5, SEQ ID NO:6 and nucleic acid sequences which hybridise to any of the foregoing sequences under stringent hybridization conditions, wherein said nucleic acid molecule encodes a glucosyltransferase polypeptide which glucosylates abscisic acid or an analogue of abscisic acid;
ii) providing conditions which facilitate the production of glucosylated abscisic acid by said cell; and
iii) isolating produced glucosylated abscisic acid from the cell or the cell-culture medium.

53. A method according to claim 52, wherein the produced glucosylated abscisic acid is the (+) abscisic acid enantiomer.

54. A method according to claim 52, wherein the produced glucosylated abscisic acid is the (−) abscisic acid enantiomer.

55. A screening method for identifying an agent able to inhibit plant growth or viability comprising:
i) providing a polypeptide encoded by a nucleic acid molecule which comprises a nucleic acid sequence selected from the group consisting of SEQ ID NO:1, SEQ ID NO:2, SEQ ID NO:3, SEQ ID NO:4, SEQ ID NO:5, SEQ ID NO:6 and nucleic acid sequences which hybridise to any of the foregoing sequences under stringent hybridization conditions, wherein said polypeptide glucosylates abscisic acid or an analogue of abscisic acid;
ii) providing at least one candidate agent;
iii) forming a preparation of (i) and (ii);
iv) providing a detectable amount of abscisic acid;
v) detecting or measuring the glucosylation activity of the polypeptide in (i) with respect to abscisic acid in (iv); and
vi) testing the effect of the agent on the growth or viability of plants.

56. A method according to claim 55, wherein said agent has herbicidal activity.

57. A method according to claim 55, wherein said polypeptide is encoded by a nucleic acid molecule consisting of a nucleic acid sequence selected from the group consisting of SEQ ID NO:1, SEQ ID NO:2, SEQ ID NO:3, SEQ ID NO:4, SEQ ID NO:5 and SEQ ID NO:6.

58. A method according to claim 55, wherein the abscisic acid is provided at between about 0.1 mM and about 2.0 mM abscisic acid.

59. A method according to claim 55, wherein said polypeptide is expressed by a cell line, and the preparation in (iii) comprises a cell culture of said cell line to which said agent has been added.

60. An agent identified by the method according to claim 55.

61. A method of testing a herbicidal agent for inhibitory activity with respect to glucosylation of abscisic acid comprising:
i) providing a transgenic cell according to claim 36 or a plant comprising said cell;
ii) applying an agent to be tested to said cell or plant;
iii) detecting or measuring the effect of the agent on growth or viability of said cell or plant;
iv) comparing the growth or viability of the cell or plant treated by application of said agent with an untreated control cell or plant; and
v) applying the agent to a non-transgenic plant or plant cell to test for efficacy.

62. A method of inhibiting growth of undesired vegetation comprising applying an agent identified by the method according to claim 55.

63. A methods of inhibiting growth of undesired vegetation comprising applying and agent identified by the method according to claim 61.

64. A method of modifying abscisic acid or an analogue thereof comprising treating the abscisic acid or abscisic acid analogue in vitro in the presence of a glucose providing moiety with a polypeptide encoded by a nucleic acid molecule which comprises a nucleic acid sequence selected from the group consisting of SEQ ID NO:1, SEQ ID NO:2, SEQ ID NO:3, SEQ ID NO:4, SEQ ID NO:5, SEQ ID NO:6 and nucleic acid sequences which hybridise to any of the foregoing sequences under stringent hybridization conditions, wherein said polypeptide is able to glucosylate the abscisic acid or abscisic acid analogue.

65. A method of testing the activity of an abscisic acid glucosyltransferase to modify a test abscisic acid analogue, said method comprising:
i) forming a preparation comprising said test analogue and an abscisic acid glucosyltransferase encoded by a nucleic acid molecule which comprises a nucleic acid sequence selected from the group consisting of SEQ ID NO:1, SEQ ID NO:2, SEQ ID NO:3, SEQ ID NO:4, SEQ ID NO:5, SEQ ID NO:6 and nucleic acid sequences which hybridise to any of the foregoing sequences under stringent hybridization conditions, wherein said glucosyltransferase glucosylates abscisic acid or an analogue of abscisic acid; and
ii) determining whether or not a glucosyl moiety is conjugated to said test abscisic acid analogue.

66. A method according to claim 65, wherein said test analogue is tested for resistance to 7′ or 8′ hydroxylation of said analogue.

67. An in vitro method for producing glucosylated abscisic acid, said method comprising:
i) providing a preparation comprising abscisic acid, a glucose moiety and a glucosyltransferase encoded by a nucleic acid molecule which comprises a nucleic acid sequence selected from the group consisting of SEQ ID NO:1, SEQ ID NO:2, SEQ ID NO:3, SEQ ID NO:4, SEQ ID NO:5, SEQ ID NO:6 and nucleic acid sequences which hybridise to any of the foregoing sequences under stringent hybridization conditions, wherein said glucosyltransferase glucosylates abscisic acid; and
ii) providing reaction conditions which facilitate the addition of at least one glucosyl moiety to abscisic acid.

68. A method according to claim 67, wherein the glucosylated abscisic acid is the (+) abscisic acid enantiomer.

69. A method according to claim 67, wherein the glucosylated abscisic acid is the (−) abscisic acid enantiomer.

70. A method for preparing (+) abscisic acid enantiomer from a racemic mixture of abscisic acid, said method comprising:
i) forming a preparation comprising a racemic mixture of abscisic acid and at least one glucosyltransferase encoded by a nucleic acid molecule which comprises a nucleic acid sequence selected from the group consisting of SEQ ID NO:1, SEQ ID NO:2, SEQ ID NO:3, SEQ ID NO:4, SEQ ID NO:5, SEQ ID NO:6 and nucleic acid sequences which hybridise to any of the foregoing sequences under stringent hybridization conditions, wherein said glucosyltransferase glucosylates abscisic acid or an analogue of abscisic acid; and
ii) subjecting said preparation to reaction conditions which facilitate the formation of a (+) abscisic acid enantiomer from said racemic mixture.

71. A method according to claim 70, wherein a glucoslyated (−) abscisic acid enantiomer formed by the method is converted to racemic abscisic acid and is added back to said preparation.

72. A method according to claim 71, wherein the glucoslyated (−) abscisic acid enantiomer is converted to a non-glucosylated (−) abscisic acid enantiomer.

73. A method according to claim 72, wherein the glucoslyated (−) abscisic acid enantiomer is converted to a non-glucosylated (−) abscisic acid enantiomer by incubation with a glucosidase.
